(19) United States
(12) Patent Application Publication
(10) Pub. No.: US 2023/0217977 A1
(43) Pub. Date: Jul. 13, 2023

(54) DIETARY CHOLINE SUPPLEMENTATION TO REDUCE TUMOR VOLUME AND ENHANCE COGNITIVE RESPONSE DURING CANCER TREATMENT

(71) Applicant: UNIVERSITY OF SOUTH FLORIDA, Tampa, FL (US)

(72) Inventors: Rex M. Philpot, Tampa, FL (US); Bethany Johns, Tampa, FL (US)

(21) Appl. No.: 18/184,618

(22) Filed: Mar. 15, 2023

Related U.S. Application Data
(63) Continuation of application No. 16/863,351, filed on Apr. 30, 2020.
(60) Provisional application No. 62/840,845, filed on Apr. 30, 2019.
(60) Provisional application No. 62/840,753, filed on Apr. 30, 2019.

(51) Int. Cl. A61K 31/14; A61K 31/704; A61K 31/675; A61P 35/00; A23L 33/00

(52) U.S. Cl. CPC A61K 31/14; A61K 31/704; A61K 31/675; A61P 35/00; A23L 33/30; A23L 33/40

(57) ABSTRACT

Described herein is a method for reducing tumor volume in a subject, the method including the steps of administering a cancer treatment (administration of at least one chemotherapeutic agent and/or application of radiation) to the subject in conjunction with the administration of choline, a choline metabolite, a choline precursor molecule, or a combination thereof. In another aspect, the disclosure relates to a method for enhancing cognitive response in a subject during cancer treatment, the method including the steps of administering cancer treatment (administration of at least one chemotherapeutic agent and/or application of radiation) to the subject in conjunction with the administration of choline, a choline metabolite, a choline precursor molecule, or a combination thereof. In still another aspect, choline, a choline metabolite, a choline precursor molecule, or a combination thereof can be administered in the form of a choline-enriched diet, a choline supplement, or a combination thereof. In some aspects, the method also enhances the effectiveness of chemotherapy.

### CROSS REFERENCE TO RELATED APPLICATIONS

This application claims priority upon U.S. provisional application Ser. Nos. 62/840,753 and 62/840,845 both filed on Apr. 30, 2019. These applications are hereby incorporated by reference in their entirety.

### STATEMENT REGARDING FEDERALLY SPONSORED RESEARCH OR DEVELOPMENT

This invention was made with U.S. Government support under grant number AT009734 awarded by the National Institutes of Health. The U.S. government has certain rights in the invention.

### BACKGROUND

Many patients report deficits of learning, memory, attention, and processing speed during and after chemotherapy. These chemotherapy-related cognitive deficits (CRCDs) are persistent and can impair day-to-day functioning for decades, adversely impacting quality of life. Many chemotherapeutic agents suppress ovarian function and decrease circulating estrogen levels, which may underlie the manifestation and persistence of CRCDs in women. Because estrogen regulates high affinity choline uptake (HACU) and HACU is the rate-limiting step for acetylcholine synthesis, chemotherapeutic agents may indirectly impair processes mediated by cholinergic systems. Both tumors and chemotherapeutic agents can elevate pro-inflammatory chemokines and cytokines, a consequence that may mediate the manifestation of CRCDs. Because acetylcholine suppresses cytokine synthesis, inhibits inflammation, and prevents tissue damage and cell death by activating α7 nicotinic acetylcholine receptors located on cytokine-producing cells, impaired HACU and acetylcholine synthesis following chemotherapy may exacerbate the adverse effects of neuroinflammation caused by tumors and chemotherapeutic agents.

What is needed is a method for increasing available choline in order to maintain cholinergic function when demand for choline is high, as during treatment with chemotherapeutic agents. It would further be advantageous if this method could activate α7 nicotinic acetylcholine receptors located on cytokine producing cells in order to attenuate pro-inflammatory processes caused by the presence of tumors, the immune response thereto, and/or treatment with chemotherapeutic agents. It would further be advantageous if the method resulted in a further reduction of tumor volume compared to treatment with chemotherapeutic agents alone. It would additionally be advantageous if the method could enhance cognitive response in subjects undergoing cancer treatment and/or could enhance the effectiveness of the chemotherapeutic agents. These needs and other needs are satisfied by the present disclosure.

### SUMMARY

In accordance with the purpose(s) of the present disclosure, as embodied and broadly described herein, the disclosure, in one aspect, relates to a method for reducing tumor volume in a subject, the method including the steps of administering a cancer treatment (administration of at least one chemotherapeutic agent and/or application of radiation) to the subject in conjunction with the administration of choline, a choline metabolite, a choline precursor molecule, or a combination thereof. In another aspect, the disclosure relates to a method for enhancing cognitive response in a subject during cancer treatment, the method including the steps of administering cancer treatment (administration of at least one chemotherapeutic agent and/or application of radiation) to the subject in conjunction with the administration of choline, a choline metabolite, a choline precursor molecule, or a combination thereof. In still another aspect, choline, a choline metabolite, a choline precursor molecule, or a combination thereof can be administered in the form of a choline-enriched diet, a choline supplement, or a combination thereof. In some aspects, the method also enhances the effectiveness of chemotherapy.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows total tumor volume in mm³ a week prior to the initiation of a 2% choline diet, at the start of a 2% choline diet, and after one week on a 2% choline diet. Tumor volume of mice on the choline diet was significantly smaller after one week on the diet.

FIG. 2 shows total tumor volume in mm³ for mice on standard (white bars) and 2% choline (gray bars) diets for a saline-only control (left) and for mice treated with cyclophosphamide (CYP) and doxorubicin (DOX). As expected, CYP+DOX treatment reduced tumor volume; additionally, for both control and CYP+DOX groups, mice on the choline-enhanced diet had lower tumor volumes than mice on a standard diet.

FIG. 3 shows high affinity choline uptake (HACU, in pmol/mg/5 min) in the striatum of mice. Left: HACU uptake is suppressed in mice on a standard diet upon administration of CYP+DOX relative to a saline control, but rescued by administration of a 2% choline diet. Inset: HACU uptake is further suppressed in mouse mammary tumor virus (MMTV) positive mice on a standard diet receiving CYP+DOX but rescued by administration of a 2% choline diet.

FIG. 4 shows HACU (in pmol/mg/5 min) in the hippocampus of mice. Left: HACU uptake is suppressed in mice on a standard diet upon administration of CYP+DOX relative to a saline control, but rescued by administration of a 2% choline diet. Inset: HACU uptake is further suppressed in mouse mammary tumor virus (MMTV) positive mice on a standard diet receiving CYP+DOX but rescued by administration of a 2% choline diet.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a choline precursor,” “a choline metabolite,” or “a chemotherapeutic agent,” include, but are not limited to, combinations or mixtures of two or more such choline precursors, choline metabolites, or chemotherapeutic agents, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference to each various individual and collective combination and permutation of these compounds may not be explicitly disclosed, each is specifically contemplated and described herein. For example, if a choline metabolite is disclosed and discussed and a number of different chemotherapeutic agents are discussed, each and every combination of choline metabolite and chemotherapeutic agent that is possible is specifically contemplated unless specifically indicated to the contrary. For example, if a class of molecules A, B, and C are disclosed, as well as a class of molecules D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered from the disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such combination is specifically contemplated and should be considered disclosed.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of choline refers to an amount that is sufficient to achieve the desired improvement in the property modulated by the formulation component, e.g. achieving the desired level of cognitive enhancement, reduction in tumor volume, or the like. The specific level in terms of wt % in a composition required as an effective amount will depend upon a variety of factors including the type and size of tumor; dosage and type of chemotherapeutic agent; age, sex, and body weight of the subject; and length of time the choline, choline metabolite, or choline precursor is administered.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human, domesticated animals such as dogs, cats, and horses, livestock such as cows and pigs, or wild animals) in need of treatment or prevention of a tumor or other cancer. “Subject” can also refer to a cell, a population of cells, a tissue, an organ, or an organism, preferably to human and constituents thereof.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Chemotherapeutic Agents

In one aspect, the methods disclosed herein involve administering at least one chemotherapeutic agent to a subject. In a further aspect, the chemotherapeutic agents disclosed herein are useful for treating tumors or cancers in a subject. In a still further aspect, various chemotherapeutic agents are envisioned for use in the methods disclosed herein.

Examples of chemotherapeutic agents include, but are not limited to, platinum compounds (e.g., cisplatin, carboplatin, oxaliplatin), alkylating agents (e.g., cyclophosphamide, ifosfamide, chlorambucil, nitrogen mustard, thiotepa, melphalan, busulfan, procarbazine, streptozocin, temozolomide, dacarbacine, bendamustine), antitumor antibiotics (e.g., daunorubicin, doxorubicin, idarubicin, epirubicin, mitoxantrone, bleomycin, mitomycin C, plicamycin, dactinomycin, amphotericin B, nystatin), taxanes (e.g., paclitaxel and docetaxel), antimetabolites (e.g., 5-fluorouracil, cytarabine, premetrexed, thioguanine, floxuridine, capecitabine, gemcitabine, and methotrexate), nucleoside analogues (e.g., fludarabine, clofarabine, cladribine, penostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vincristine, vindesine, vinorelbine, and vinblastine), tyrosine kinase inhibitors (e.g., imatinib, dasatinib, nilotinib, sorafenib, sunitinib), monoclonal antibodies (e.g., rituximab, cetuximab, panetumumab, tositumomab, trastuzumab, alemtuzumab, gemtuzumab, ozogamicin, bevacizumab), nitrosoureas (e.g., carmustine, fotemustine, and lumustine), enzymes (e.g., L-asparaginase), biological agents (e.g., interferons and interleukins), hexamethylmelamine, mitotane, angiogenesis inhibitors (e.g., thalidomide, lenalidomide), steroids (e.g., prednisone, dexamethasone, betulinic acid, testosterone, estrogen, progesterone, and prednisolone), hormonal agents (e.g., tamoxifen, faloxifene, leuprolide, bicalutamide, granisetron, flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, oxaprozin), selective cyclooxygenase-2 (COX-2) inhibitors, diazepam, propofol, 2,3-mercaptopropanol, or any combination thereof. In one aspect, the chemotherapeutic agent is cyclophosphamide, doxorubicin, or a combination thereof.

In any of these aspects, the ordinarily-skilled artisan will be able to select a dose and dosage form of the chemotherapeutic agent based on tumor type; tumor size; patient's age, sex, body weight, and overall health; and the like.

### Radiation

In one aspect, the methods disclosed herein involve applying radiation to a subject for cancer treatment. Radiation treatment alone and in combination with chemotherapy is a traditional regimen. In one aspect, the cancer patient can be exposed to radiation via a linear accelerator, betatron, or microtron, which is referred to as teletherapy. In another aspect, radioactive sources are placed within the body of the patient so that the radiation source is close to the tissue to be treated.

### Choline

In one aspect, the methods disclosed herein involve administering choline, a choline metabolite, a choline precursor compound, or a combination thereof to a subject.

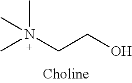

As used herein, choline is a water-soluble quaternary ammonium compound that is useful as a precursor for other essential components of the cell including, but not limited to, phospholipids, neurotransmitters, and osmoregulators. In another aspect, some choline metabolites such as, for example, betaine, may serve as methyl group donors for the biosynthesis of S-adenosylmethionine.

In some aspects, the presence of cancer and/or a tumor may deplete blood levels of choline through various mechanisms. In other aspects, the administration of one or more chemotherapeutic agents can also lead to reduced blood levels of choline. Thus, in one aspect, it is desirable to replenish the level of choline available to a subject in need thereof. In one aspect, provided herein is a method for increasing choline levels.

In any of these aspects, choline supplementation can be accompanied by folate supplementation. In one aspect, folate can be an important methyl group donor for synthesis of S-adenosylmethionine.

In one aspect, reduced choline intake and/or choline deficiency may cause muscle damage. In another aspect, choline deficiency can affect the liver and may cause liver damage, nonalcoholic fatty liver disease, or another condition. In another aspect, persons receiving nutrition parenterally may be at risk for choline deficiency.

In some aspects, choline may be useful for reducing blood pressure, favorably altering lipid profiles, and reducing levels of homocysteine in the blood. In other aspects, choline may be important for protecting the structural integrity of neurons and may assist in the treatment of dementia or cognitive decline that is either temporary (as induced, for example, by chemotherapy) or associated with a condition such as, for example, Alzheimer's disease, Parkinson's disease, or the like. In still another aspect, choline supplementation may improve retinal function in glaucoma patients and/or may be useful for limiting neuron damage in stroke patients. In any of these aspects, inadequate choline intake may exacerbate the problems that choline supplementation may be useful for treating or preventing.

In one aspect, the method includes the step of feeding the subject a diet rich in choline. In a further aspect, foods rich in choline include, but are not limited to, eggs; meat such as, for example, beef and beef liver; poultry such as, for example, chicken; fish such as, for example, cod and/or tuna; shellfish such as, for example, scallops and/or shrimp; cruciferous vegetables such as, for example, broccoli, brussels sprouts, cabbage, and cauliflower; legumes such as, for example, peas, kidney beans, snap beans, and peanuts; dairy products such as, for example, milk, yogurt, and cottage cheese; wheat and wheat products including, but not limited to, pasta and wheat germ; beets; spinach; rice; red potatoes; shiitake mushrooms; quinoa; sunflower seeds; tangerines; kiwi fruits; carrots; apples; and lecithin appearing in processed foods.

In a further aspect, in the method disclosed herein, the choline-rich diet can be from about 1 to about 5% choline, or can be about 1, 1.5, 2, 2.5, 3, 3.5, 4, 4.5, or about 5% choline, or a combination of any of the foregoing values, or a range encompassing any of the foregoing values. In one aspect, the choline-rich diet is about 2% choline.

In some aspects, it may be difficult to increase dietary choline to the levels disclosed herein. Further in these aspects, choline supplements can be provided to the subject in order to increase choline intake. In one aspect, the choline supplements are provided instead of a choline-rich diet. In an alternative aspect, both choline supplements and a choline-rich diet are provided.

In one aspect, the choline supplement can take the form of choline, a choline salt, a choline metabolite, or a choline precursor compound. In a further aspect, the choline salt can be selected from choline hydroxide, choline chloride, choline bitartrate, choline magnesium trisalicylate, or a combination thereof. In one aspect, the choline salt is choline bitartrate.

In a further aspect, the choline supplement can be provided as an oral supplement such as, for example, a liquid, syrup, suspension, tablet, capsule, caplet, functional food, or other dosage form. In an alternative aspect, the choline supplement can be provided to the subject as an injection or intravenously. In any of these aspects, the choline supplement can be administered once or twice daily, with or without meals. In some aspects, the choline supplement can be a commercially-available product. In another aspect, when the subject is a non-human mammal, the choline-rich diet can be a commercial lab diet or other commercial product.

In one aspect, the typical upper intake of choline for adults undergoing supplementation can be from about 3000 to about 5000 mg per day, or can be about 3000, 3500, 4000, 4500, or about 5000 mg per day, or a combination of any of the foregoing values, or a range encompassing any of the foregoing values. In one aspect, typical upper intake of choline is about 3500 mg per day.

### Choline Metabolites

In some aspects, choline supplements or a choline-rich diet can incorporate one or more choline metabolites. In a further aspect, in some instances, choline metabolites are not processed into choline directly but can be used by the body as-is, thereby freeing up other choline to be processed into needed metabolites that may not be present, or that are not present in sufficient quantities.

In one aspect, the choline metabolite can be selected from acetylcholine, betaine, glycerophosphocholine, phosphatidylcholine, sphingomyelin, lysophosphatidylcholine, citicoline (also known as cytidine 5′-diphosphocholine or CDP-choline), L-α glycerylphosphorylcholine (also known as alpha-GPC or GPC choline), another choline metabolite, or a combination thereof.

### Choline Precursor Compounds

In other aspects, choline supplements or a choline-rich diet can incorporate one or more choline precursor compounds. Without wishing to be bound by theory, a choline precursor compound can be metabolized by the body to produce choline and choline metabolites. In one aspect, the choline precursor compound can be selected from L-serine, ethanolamine, a phosphatidylcholine, another choline precursor compound, or a combination thereof.

### Choline Kinase Inhibitors

In certain aspects, an optional choline kinase inhibitor can be co-administered to the subject. Choline kinase inhibitors, as used herein, relate to any compound capable of causing a decrease in the ChoK activity, including those compounds which prevent expression of the ChoK gene, leading to reduced ChoK mRNA or protein levels as well as compounds that inhibit ChoK causing a decrease in the activity of the enzyme. In one aspect, the choline kinase inhibitors are specific for choline kinase alpha.

Compounds leading to reduced ChoK mRNA levels can be identified using standard assays for determining mRNA expression levels such as RT-PCR, RNA protection analysis, Northern blot, in situ hybridization, microarray technology and the like.

Compounds leading to reduced ChoK protein levels can be identified using standard assays for determining protein expression levels such as Western-blot or Western transfer, ELISA (enzyme-linked immunosorbent assay), RIA (radioimmunoassay), competitive EIA (competitive enzyme immunoassay), DAS-ELISA (double antibody sandwich ELISA), immunocytochemical and immunohistochemical techniques, techniques based on the use of protein biochips or microarrays which include specific antibodies or assays based on colloidal precipitation in formats such as dipsticks.

### Method for Reducing Tumor Volume

Disclosed herein are methods for reducing tumor volume in a subject. In one aspect, the subject can be a mammal such as, for example, a mouse or human, having at least one tumor. In one aspect, the method involves co-administering at least one chemotherapeutic agent and choline, a choline metabolite, a choline precursor compound, or a combination thereof to the subject. In another aspect, the method involves applying radiation to the subject and administering choline, a choline metabolite, a choline precursor compound, or a combination thereof to the subject. In another aspect, the method involves applying radiation to the subject and co-administering at least one chemotherapeutic agent and choline, a choline metabolite, a choline precursor compound, or a combination thereof to the subject.

In one aspect, reducing tumor volume refers to a decrease in the size of a tumor, wherein the tumor volume is measured before and after treatment according to the method disclosed herein.

In one aspect, tumor volume can be reduced by from at least 25% to at least 30% or more compared to tumor volume prior to treatment using the methods disclosed herein (i.e., increased choline intake coupled with treatment with at least one chemotherapeutic agent and/or radiation), or can be reduced by 25, 26, 27, 28, 29, 30%, or more compared to tumor volume prior to treatment.

In another aspect, although tumor volume may decrease with only chemotherapy and/or radiation without administration of choline, a choline metabolite, a choline precursor compound, or a combination thereof, tumor volume can be further decreased by performance of the methods disclosed herein. In a further aspect, when choline, a choline metabolite, a choline precursor compound, or a combination thereof, are administered to the subject in conjunction with the at least one chemotherapeutic agent and/or radiation, tumor volume may be reduced by at least an additional 20% to 25% or greater, or by at least 20, 21, 22, 23, 24, or 25% or greater, compared to administration of the chemotherapeutic agent and/or radiation alone. Thus, in this aspect, supplementation with choline, a choline metabolite, a choline precursor compound, or combination thereof, through diet or otherwise, can enhance the effectiveness (i.e., provide a greater reduction in tumor size) of conventional cancer treatments such as, for example, administration of chemotherapeutic agents and/or radiation.

In one aspect, the choline, choline metabolite, choline precursor compound, or combination thereof is administered to the subject prior to the administration of the at least one chemotherapeutic agent and/or exposure to radiation. In another aspect, the choline, choline metabolite, choline precursor compound, or combination thereof is administered to the subject simultaneously with the at least one chemotherapeutic agent and/or exposure to radiation. In still another aspect, administration of the choline, choline metabolite, choline precursor compound, or combination thereof, whether in the form of a choline-rich diet, a supplement, or a combination thereof, is begun at least one week or at least two weeks prior to administration of the at least one chemotherapeutic agent and/or exposure to radiation. Further in this aspect, the choline, choline metabolite, choline precursor compound, or combination thereof can be administered daily or can be administered multiple times per day (e.g., a choline-rich diet would be consumed at each meal) for the entire period leading up to administration of the at least one chemotherapeutic agent and/or exposure to radiation.

In some aspects, administration of the at least one chemotherapeutic agent and/or exposure to radiation can be ongoing over a period of days, weeks, or months. Further in these aspects, the choline, choline metabolite, choline precursor compound, or combination thereof can be administered during the entire treatment period wherein the chemotherapeutic agent and/or exposure to radiation is administered to the subject. In another aspect, the choline, choline metabolite, choline precursor compound, or combination thereof is additionally administered to the subject for a period of time following completion of treatment with the chemotherapeutic agent and/or exposure to radiation.

### Method for Enhancing Cognitive Response

In one aspect, disclosed herein is a method for enhancing cognitive response in a subject in need thereof. In a further aspect, the subject can be a mammal such as, for example, a mouse or human, experiencing cognitive decline caused by, for example, the presence of at least one tumor, a reduction in plasma choline levels, and/or undergoing cancer treatment (chemotherapy and/or exposure to radiation) or other treatment for one of the aforementioned conditions. In one aspect, the method involves administering choline, a choline metabolite, a choline precursor compound, or a combination thereof to the subject in conjunction with cancer treatment (chemotherapy and/or exposure to radiation).

In one aspect, the presence of a tumor and/or treatment with a chemotherapeutic agent and/or exposure to radiation may lead to a decrease in blood or plasma choline. In a further aspect, this decrease may result in a reduction of acetylcholine synthesis. In a still further aspect, acetylcholine is a neurotransmitter that can cause the contraction of skeletal muscles as well as activating certain endocrine functions. In another aspect, acetylcholine is also important for feeling pain, for learning, for regulation of rapid eye movement (REM) sleep, and for memory formation, including working memory. Still further in this aspect, restoring acetylcholine synthesis can protect cognitive function and/or treat chemotherapy-induced cognitive deficits in subjects.

In one aspect, high affinity choline uptake (HACU) is a rate-limiting step for acetylcholine synthesis. Further in this aspect, a reduction in choline uptake can undermine functions that are mediated by acetylcholine. In one aspect, placing a subject on a high-choline diet and/or supplementing with choline, a choline precursor compound, or a choline metabolite during cancer treatment can protect these cognitive processes.

In one aspect, cognitive response as used herein refers to learning, memory, attention, processing speed, and/or other mental processes. In one aspect, presence of a tumor and/or chemotherapy can lead to chemotherapy-related cognitive deficits (CRCDs) in these processes. In a still further aspect, CRCDs can impair day-to-day functioning for decades after chemotherapy is completed and thus adversely impact quality of life.

In another aspect, tumors and/or cancer treatment (chemotherapy and/or exposure to radiation) can increase inflammation, increase levels of cytokines and/or chemokines, and or can cause tissue damage.

In one aspect, inflammation can lead to a decrease in cognitive function. Although inflammation can facilitate the healing process, in one aspect, prolonged inflammation can result in tissue damage. In another aspect, inflammation may be associated with neurodegeneration, impaired neurogenesis, atherosclerosis, chronic diseases, or the like.

In another aspect, systemic immune activation in the form of chronic or long-term inflammation can be associated with elevated serum levels of inflammatory chemokines including, but not limited to, cytokines such as, for example, interleukin 6, tumor necrosis factor alpha, C-reactive protein, and the like. In a further aspect, these cytokines can lead to impairment of overall cognition as well as impairments in specific cognitive functions including, but not limited to, processing speed, memory, and executive function. In another aspect, inflammation can lead to hippocampal atrophy and other changes to the brain associated with cognitive deficits.

In one aspect, “enhanced cognitive response” as disclosed herein refers to an improvement in a cognitive or mental process such as, for example, learning, memory, attention, processing speed, or another mental process, including, but not limited to, processes and functions mediated by HACU or acetylcholine. In a further aspect, enhanced cognitive response can be measured by assessing a subject prior to performing the method disclosed herein and also assessing the same subject after performing the method disclosed herein by a known technique such as, for example, patient self-reporting, performance of memory, learning, and/or concentration-oriented tasks and independent assessment of results thereof, functional MRI or other imaging method designed to assess blood flow to various areas of the brain associated with neuronal activation for a given task, or another method.

In one aspect, the method disclosed herein results in enhanced cognitive response relative to treatment with cancer treatment (chemotherapy and/or exposure to radiation) alone and/or relative to no treatment at all. In another aspect, the method disclosed herein results in an improvement of HACU of from at least 5% to at least 15% or more compared to HACU measured in the subject prior to beginning the treatment, or of at least 5, 6, 7, 8, 9, 10, 11, 12, 13, 14, 15%, or greater, or a combination of any of the foregoing values, or a range encompassing any of the foregoing values.

In one aspect, the choline, choline metabolite, choline precursor compound, or combination thereof is administered to the subject prior to the administration of the at least one chemotherapeutic agent and/or exposure to radiation. In another aspect, the choline, choline metabolite, choline precursor compound, or combination thereof is administered to the subject simultaneously with the at least one chemotherapeutic agent and/or exposure to radiation. In still another aspect, administration of the choline, choline metabolite, choline precursor compound, or combination thereof, whether in the form of a choline-rich diet, a supplement, or a combination thereof, is begun at least one week or at least two weeks prior to administration of the at least one chemotherapeutic agent and/or exposure to radiation. Further in this aspect, the choline, choline metabolite, choline precursor compound, or combination thereof can be administered daily or can be administered multiple times per day (e.g., a choline-rich diet would be consumed at each meal) for the entire period leading up to administration of the at least one chemotherapeutic agent and/or exposure to radiation.

In some aspects, administration of the at least one chemotherapeutic agent and/or exposure to radiation can be ongoing over a period of days, weeks, or months. Further in these aspects, the choline, choline metabolite, choline precursor compound, or combination thereof can be administered during the entire cancer treatment period. In another aspect, the choline, choline metabolite, choline precursor compound, or combination thereof is additionally administered to the subject for a period of time following completion of cancer treatment.

Aspects

Aspect 1: A method for reducing tumor volume in a subject during cancer treatment, the method comprising administering choline, a choline metabolite, a choline precursor compound, or a combination thereof to the subject, wherein the cancer treatment comprises administering a chemotherapeutic agent to the subject, exposing the subject to radiation, or a combination thereof.

Aspect 2: The method of aspect 1, wherein tumor volume is reduced by at least 25% relative to the volume of the tumor prior to performing the method.

Aspect 3: The method of aspect 1, wherein tumor volume is reduced by at least 20% relative to tumor volume of cancer treatment without the administration of choline, a choline metabolite, a choline precursor compound, or a combination thereof.

Aspect 4: The method in any one of aspects 1 to 3, wherein the choline, choline metabolite, choline precursor compound, or combination thereof is administered prior to cancer treatment.

Aspect 5: The method in any one of aspects 1 to 3, wherein the choline, choline metabolite, choline precursor compound, or combination thereof is administered simultaneously with the cancer treatment.

Aspect 6: The method in any one of aspects 1 to 5, wherein the choline, choline metabolite, choline precursor compound, or combination thereof is administered as a supplement, as part of a choline-rich diet, or a combination thereof.

Aspect 7: The method of aspect 6, wherein the choline-rich diet comprises at least 2% choline, choline metabolite, choline precursor compound, or combination thereof.

Aspect 8: The method in any one of aspects 1 to 7, wherein the choline metabolite comprises acetylcholine, betaine, glycerophosphocholine, phosphatidylcholine, sphingomyelin, lysophosphatidylcholine, citicoline, L-a glycerylphosphorylcholine, or a combination thereof.

Aspect 9: The method in any one of aspects 1 to 7, wherein the choline precursor compound comprises L-serine, ethanolamine, a phosphatidylcholine, or a combination thereof.

Aspect 10: The method in any one of aspects 1 to 9, wherein the at least one chemotherapeutic agent comprises doxorubicin, cyclophosphamide, or a combination thereof.

Aspect 11: A method for enhancing cognitive response in a subject during cancer treatment, the method comprising administering choline, a choline metabolite, a choline precursor compound, or a combination thereof to the subject, wherein the cancer treatment comprises administering a chemotherapeutic agent to the subject, exposing the subject to radiation, or a combination thereof.

Aspect 12: The method of aspect 11, wherein the method increases high affinity choline uptake in the brain by at least 5% relative to cancer treatment without choline, a choline metabolite, a choline precursor compound, or a combination thereof.

Aspect 13: The method of aspect 11, wherein the method inhibits inflammation, reduces levels of cytokines or chemokines, prevents tissue damage, or a combination thereof, relative to cancer treatment without choline, a choline metabolite, a choline precursor compound, or a combination thereof.

Aspect 14: The method in any one of aspects 11 to 13, wherein the choline, choline metabolite, choline precursor compound, or combination thereof is administered prior to cancer treatment.

Aspect 15: The method in any one of aspects 11 to 13, wherein the choline, choline metabolite, choline precursor compound, or combination thereof is administered simultaneously with cancer treatment.

Aspect 16: The method in any one of aspects 11 to 15, wherein the choline, choline metabolite, choline precursor compound, or combination thereof is administered as a supplement, as part of a choline-rich diet, or a combination thereof.

Aspect 17: The method of aspect 16, wherein the choline-rich diet comprises at least 2% choline, choline metabolite, choline precursor compound, or combination thereof.

Aspect 18: The method in any one of aspects 11 to 17, wherein the choline metabolite comprises acetylcholine, betaine, glycerophosphocholine, phosphatidylcholine, sphingomyelin, lysophosphatidylcholine, citicoline, L-a glycerylphosphorylcholine, or a combination thereof.

Aspect 19: The method in any one of aspects 11 to 17, wherein the choline precursor compound comprises L-serine, ethanolamine, a phosphatidylcholine, or a combination thereof.

Aspect 20: The method in any one of aspects 11 to 19, wherein the at least one chemotherapeutic agent comprises doxorubicin, cyclophosphamide, or a combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Reduction in Tumor Volume and Enhancement of Cancer Treatment Effectiveness

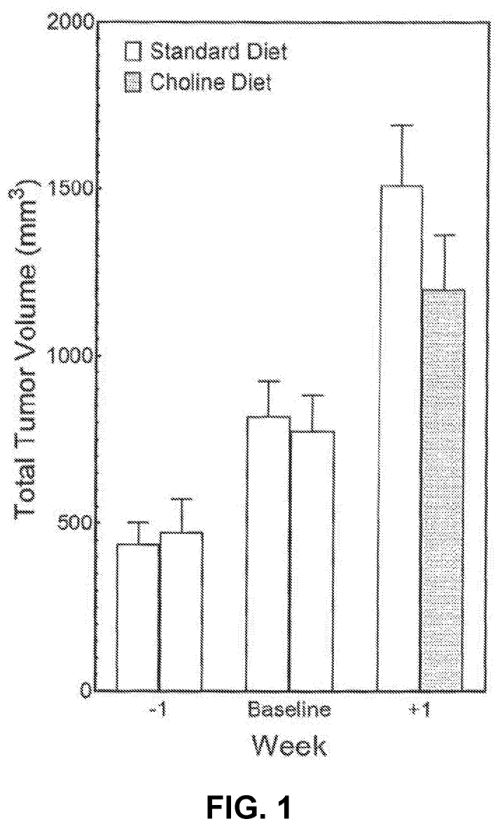

Tumor-bearing mice were provided with a diet consisting of 2% choline for all studies (Teklad Customized Diets from ENVIGO, Inc.). Total average tumor volume of tumor-bearing mice on the choline-enhanced diet was 23% smaller than total average tumor volume for mice maintained on a standard diet (FIG. 1).

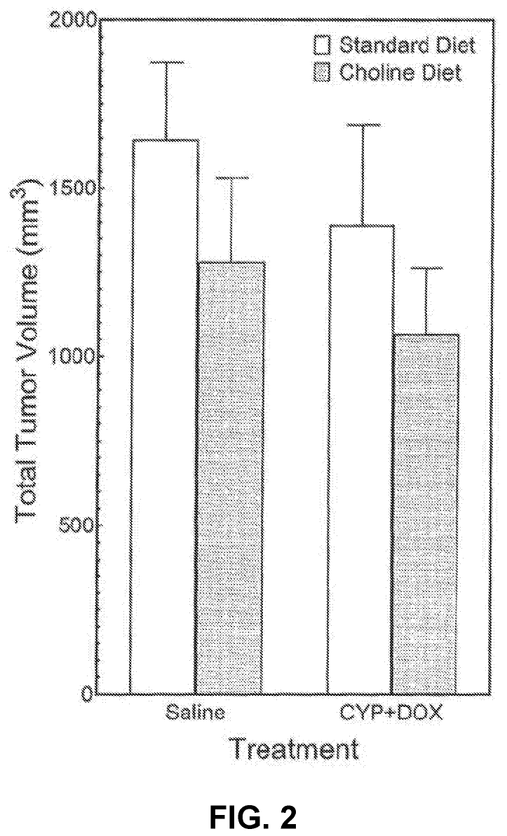

Following a single administration of doxorubicin and cyclophosphamide, tumor-bearing mice on a choline-enhanced diet exhibited total tumor volumes that were 28% smaller than mice on standard diets but receiving the same chemotherapy (FIG. 2).

### Example 2: NACU Uptake and Cognitive Response

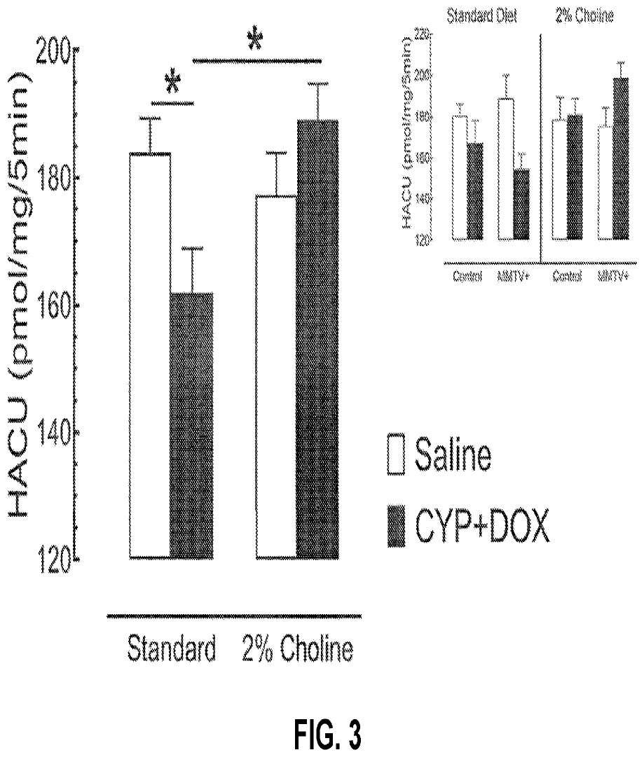

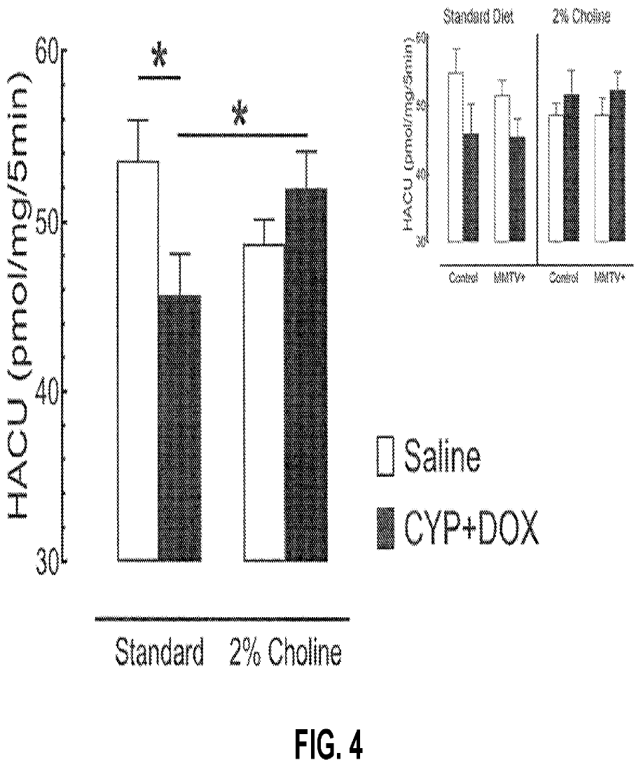

Chemotherapeutic agents such as doxorubicin and cyclophosphamide reduced high affinity choline uptake (HACU) in the brain by 8-18%. Mouse mammary tumor virus-positive (MMTV+) mice were provided with a diet consisting of 2% choline for all studies (Teklad Customized Diets from ENVIGO, Inc.) prior to administration of chemotherapy. Provision of this diet prevented reduction in HACU in both the striatum (FIG. 3) and hippocampus (FIG. 4).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method for reducing tumor volume in a subject during cancer treatment, the method comprising administering choline, a choline metabolite, a choline precursor compound, or a combination thereof to the subject, wherein the cancer treatment comprises administering a chemotherapeutic agent to the subject, exposing the subject to radiation, or a combination thereof.

2. The method of claim 1, wherein tumor volume is reduced by at least 25% relative to the volume of the tumor prior to performing the method.

3. The method of claim 1, wherein tumor volume is reduced by at least 20% relative to tumor volume of cancer treatment without the administration of choline, a choline metabolite, a choline precursor compound, or a combination thereof.

4. The method of claim 1, wherein the choline, choline metabolite, choline precursor compound, or combination thereof is administered prior to cancer treatment.

5. The method of claim 1, wherein the choline, choline metabolite, choline precursor compound, or combination thereof is administered simultaneously with the cancer treatment.

6. The method of claim 1, wherein the choline, choline metabolite, choline precursor compound, or combination thereof is administered as a supplement, as part of a choline-rich diet, or a combination thereof.

7. The method of claim 6, wherein the choline-rich diet comprises at least 2% choline, choline metabolite, choline precursor compound, or combination thereof.

8. The method of claim 1, wherein the choline metabolite comprises acetylcholine, betaine, glycerophosphocholine, phosphatidylcholine, sphingomyelin, lysophosphatidylcholine, citicoline, L-a glycerylphosphorylcholine, or a combination thereof.

9. The method of claim 1, wherein the choline precursor compound comprises L-serine, ethanolamine, a phosphatidylcholine, or a combination thereof.

10. The method of claim 1, wherein the at least one chemotherapeutic agent comprises doxorubicin, cyclophosphamide, or a combination thereof.

11. A method for enhancing cognitive response in a subject during cancer treatment, the method comprising administering choline, a choline metabolite, a choline precursor compound, or a combination thereof to the subject, wherein the cancer treatment comprises administering a chemotherapeutic agent to the subject, exposing the subject to radiation, or a combination thereof.

12. The method of claim 11, wherein the method increases high affinity choline uptake in the brain by at least 5% relative to cancer treatment without choline, a choline metabolite, a choline precursor compound, or a combination thereof.

13. The method of claim 11, wherein the method inhibits inflammation, reduces levels of cytokines or chemokines, prevents tissue damage, or a combination thereof, relative to cancer treatment without choline, a choline metabolite, a choline precursor compound, or a combination thereof.

14. The method of claim 11, wherein the choline, choline metabolite, choline precursor compound, or combination thereof is administered prior to cancer treatment.

15. The method of claim 11, wherein the choline, choline metabolite, choline precursor compound, or combination thereof is administered simultaneously with cancer treatment.

16. The method of claim 11, wherein the choline, choline metabolite, choline precursor compound, or combination thereof is administered as a supplement, as part of a choline-rich diet, or a combination thereof.

17. The method of claim 16, wherein the choline-rich diet comprises at least 2% choline, choline metabolite, choline precursor compound, or combination thereof.

18. The method of claim 11, wherein the choline metabolite comprises acetylcholine, betaine, glycerophosphocholine, phosphatidylcholine, sphingomyelin, lysophosphatidylcholine, citicoline, L-a glycerylphosphorylcholine, or a combination thereof.

19. The method of claim 11, wherein the choline precursor compound comprises L-serine, ethanolamine, a phosphatidylcholine, or a combination thereof.

20. The method of claim 11, wherein the at least one chemotherapeutic agent comprises doxorubicin, cyclophosphamide, or a combination thereof.
